(19) United States
(12) Patent Application Publication
(10) Pub. No.: US 2005/0070599 A1
(43) Pub. Date: Mar. 31, 2005

(54) Crystal forms of 4-[4-(4-fluorophenoxy)benzenesulfonylamino]-tetrahydropyran-4-carboxylic acid hydroxyamide

(76) Inventors: Marcus Douglas Ewing, Colchester, CT (US); Zheng Jane Li, Quaker Hill, CT (US); Lawrence Alan Reiter, Mystic, CT (US); Derek Lawrence Tickner, Waterford, CT (US)

Correspondence Address: WARNER-LAMBERT COMPANY, 2800 PLYMOUTH RD, ANN ARBOR, MI, 48105, US

(21) Appl. No.: 10/853,834

(22) Filed: May 26, 2004

Related U.S. Application Data
(60) Provisional application No. 60/475,135, filed on May 30, 2003.

(51) Int. Cl. A61K 31/35

(52) U.S. Cl. 514/459; 549/424

(57) ABSTRACT

The present invention relates to a new crystal form of the compound 4-[4-(4-fluorophenoxy)benzenesulfonylamino]-tetrahydropyran-4-carboxylic acid hydroxyamide, combinations of forms of that compound, compositions containing one or more forms of the compound, processes for preparing forms of the compound and compositions containing one or more of them, and the use of one or more forms of the compound and compositions containing one or more of them in treating medical disorders. The invention also provides a new form of the compound 4-[4-(4-fluorophenoxy)benzenesulfonylamino]-tetrahydropyran-4-carboxylic acid hydroxyamide which is referred to as Form B.

### CROSS-REFERENCE TO RELATED APPLICATIONS

This application claims priority from U.S. Provisional Patent Application No. 60/475,135 filed May 30, 2003.

### FIELD OF THE INVENTION

The present invention relates to crystal forms of the compound 4-[4-(4-fluorophenoxy)benzenesulfonylamino]-tetrahydropyran-4-carboxylic acid hydroxyamide, combinations of forms of that compound, compositions containing one or more forms of the compound, processes for preparing forms of the compound and compositions containing one or more of them, and the use of one or more forms of the compound and compositions containing one or more of them in treating medical disorders.

### BACKGROUND OF THE INVENTION

The class of compounds referred to as (4-arylsulfonylamino)-tetrahydropyran-4-carboxylic acid hydroxamide compounds and derivatives thereof are disclosed and claimed generally in Reiter, U.S. Pat. No. 6,087,392 (“Reiter”), the disclosure of which is incorporated herein by reference in its entirety. That class of compounds are known as having the ability to inhibit of zinc metalloendopeptidases and, in particular, the ability to inhibit zinc metalloendopeptidases that belong to the matrix metalloproteinase (also known as “MMP” or matrixin), and inhibit reprolysin (also known as adamylsin or “ADAMs”) subfamilies of the metzincins. The compounds may be used in compositions for treatment of a wide variety of conditions. One of the (4-arylsulfonylamino)-tetrahydropyran-4-carboxylic acid hydroxamide derivatives that is disclosed and claimed in Reiter is 4-[4-(4-fluorophenoxy)benzenesulfonylamino]-tetrahydropyran-4-carboxylic acid hydroxyamide, which has the structure:

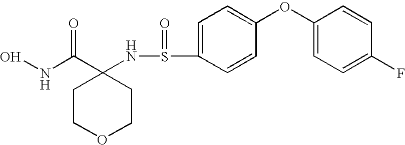

In Example 1(F) of Reiter, the preparation of a particular form of crystalline 4-[4-(4-fluorophenoxy)benzenesulfonylamino]-tetrahydropyran-4-carboxylic acid hydroxyamide, which will be referred to hereinafter as “Form A” of that compound, is disclosed. Example 1(F) also discloses specific physical characteristics possessed by Form A, such as melting point, ¹HNMR, and ¹³CNMR data.

### SUMMARY OF THE INVENTION

The present invention relates to the compound 4-[4-(4-fluorophenoxy)benzenesulfonylamino]-tetrahydropyran-4-carboxylic acid hydroxyamide and, more particularly, polymorphic forms of that compound. In one embodiment, the present invention provides a new crystalline form of the compound 4-[4-(4-fluorophenoxy)benzenesulfonylamino]-tetrahydropyran-4-carboxylic acid hydroxyamide which is referred to hereinafter as “Form B” of the compound and which possesses physical characteristics as well as other features and attributes, such as an enhanced stability, that distinguish it from Form A of 4-[4-(4-fluorophenoxy)benzenesulfonylamino]-tetrahydropyran-4-carboxylic acid hydroxyamide.

The present invention also provides pharmaceutical compositions that include Form B and/or Form A. The compositions may be administered in a variety of ways, alone or in combination with other pharmaceutical compounds or compositions, for treating a patient suffering from a medical disorder that will respond to the administration of Form B and/or Form A alone or in combination with another compound. The method includes the step of administering an effective amount of Form B and/or Form A to a patient in need thereof.

In addition, the present invention provides processes for preparing Form B and Form A, and methods of preparing compositions containing Form B and/or Form A.

### DETAILED DESCRIPTION OF THE INVENTION

New crystal Form B of 4-[4-(4-fluorophenoxy)benzenesulfonylamino]-tetrahydropyran-4-carboxylic acid hydroxyamide possesses the ability to treat the disorders, such as inflammatory diseases, that Form A may be used to treat. The treatment of such disorders may be characterized by the inhibition of zinc metalloendopeptidases matrix metalloproteinases or other metalloproteinases involved in matrix degradation. Thus, Form B and/or Form A of the compound 4-[4-(4-fluorophenoxy)benzenesulfonylamino]-tetrahydropyran-4-carboxylic acid hydroxyamide may be used to regulate one or more of the MMP subfamily of enzymes such as MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, and MMP-20. Form B and/or Form A of 4-[4-(4-fluorophenoxy)benzenesulfonylamino]-tetrahydropyran-4-carboxylic acid hydroxyamide may also be used to regulate a mammalian reprolysin (such as aggrecanase or ADAM's TS-1, 10, 12, 15 and 17, most preferably ADAM-17) in a mammal, including a human.

In all methods in which Form B and/or Form A are administered to a patient in need thereof, or to a patient as a prophylactic therapy, the method includes the step of administering an effective amount of Form B and/or Form A of 4-[4-(4-fluorophenoxy)benzenesulfonylamino]-tetrahydropyran-4-carboxylic acid hydroxyamide. The method may further include the step of identifying a patient in need of treatment with Form B and/or Form A of 4-[4-(4-fluorophenoxy)benzenesulfonylamino]-tetrahydropyran-4-carboxylic acid hydroxyamide.

More particularly, Form B and Form A of the 4-[4-(4-fluorophenoxy)benzenesulfonylamino]-tetrahydropyran-4-carboxylic acid hydroxyamide compound are each useful in treating the conditions disclosed in Reiter, such as a condition selected from the group consisting of arthritis (including osteoarthritis and rheumatoid arthritis), inflammatory bowel disease, Crohn's disease, emphysema, chronic obstructive pulmonary disease, Alzheimer's disease, organ transplant toxicity, cachexia, allergic reactions, allergic contact hypersensitivity, cancer, tissue ulceration, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), congestive heart failure, myocardial infarction, stroke, cerebral ischemia, head trauma, spinal cord injury, neuro-degenerative disorders (acute and chronic), autoimmune disorders, Huntington's disease, Parkinson's disease, migraine, depression, peripheral neuropathy, pain, cerebral amyloid angiopathy, nootropic or cognition enhancement, amyotrophic lateral sclerosis, multiple sclerosis, ocular angiogenesis, corneal injury, macular degeneration, abnormal wound healing, burns, diabetes, tumor invasion, tumor growth, tumor metastasis, corneal scarring, scleritis, AIDS, sepsis, septic shock and other diseases characterized by metalloproteinase activity and other diseases characterized by mammalian reprolysin activity in a mammal, including a human.

Crystal Form B and/or Form A of 4-[4-(4-fluorophenoxy)benzenesulfonylamino]-tetrahydropyran-4-carboxylic acid hydroxyamide may be administered in the form of a pharmaceutical composition comprising at least one of Form B and Form A together with a pharmaceutically acceptable vehicle or diluent. Thus, Form A and Form B can be administered individually or together in any conventional oral, parenteral, rectal or transdermal dosage form.

For oral administration, a pharmaceutical composition can take the form of solutions, suspensions, tablets, pills, capsules, powders, and the like.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof. In the case of animals, they are advantageously contained in an animal feed or drinking water in a concentration of 5-5000 ppm, preferably 25 to 500 ppm.

For parenteral administration (intramuscular, intraperitoneal, subcutaneous and intravenous use) a sterile injectable solution of the active ingredient is usually prepared. Solutions of Form B and/or Form A in either sesame or peanut oil or in aqueous propylene glycol may be employed. The aqueous solutions should be suitably adjusted and buffered, preferably at a pH of greater than 8, if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art. In the case of animals, Form B and/or Form A can be administered intramuscularly or subcutaneously at dosage levels of about 0.1 to 50 mg/kg/day, advantageously 0.2 to 10 mg/kg/day given in a single dose or up to 3 divided doses.

For topical ocular administration, direct application to the affected eye may be employed in the form of a formulation as eyedrops, aerosol, gels or ointments, or can be incorporated into collagen (such as poly-2-hydroxyethylmethacrylate and co-polymers thereof), or a hydrophilic polymer shield. The materials can also be applied as a contact lens or via a local reservoir or as a subconjunctival formulation.

For intraorbital administration a sterile injectable solution of the active ingredient(s) is usually prepared. Solutions of a therapeutic compound of the present invention in an aqueous solution or suspension (particle size less than 10 micron) may be employed. The aqueous solutions should be suitably adjusted and buffered, preferably at a pH between 5 and 8, if necessary and the liquid diluent first rendered isotonic. Small amounts of polymers can be added to increase viscosity or for sustained release (such as cellulosic polymers, Dextran, polyethylene glycol, or alginic acid). These solutions are suitable for intraorbital injection purposes. The preparation of all these solutions under sterile conditions is readily accomplished by standard pharmaceutical techniques well known to those skilled in the art In the case of animals, compounds can be administered intraorbitally at dosage levels of about 0.1 to 50 mg/kg/day, advantageously 0.2 to 10 mg/kg/day given in a single dose or up to 3 divided doses.

The active compound(s) of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of Form B and/or Form A of 4-[4-(4-fluorophenoxy)benzenesulfonylamino]-tetrahydropyran-4-carboxylic acid hydroxyamide and a suitable powder base such as lactose or starch.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975).

Pharmaceutical compositions according to the invention may contain for example 0.0001%-95% of the compound(s) of this invention. In any event, the composition or formulation to be administered will contain a quantity of Form B and/or Form A in an amount effective to treat the disease/condition of the subject being treated, such as the more particular amounts set forth herein.

In addition, when the compound 4-[4-(4-fluorophenoxy)benzenesulfonylamino]-tetrahydropyran-4-carboxylic acid hydroxyamide forms metabolites, hydrates, or solvates, they are also within the scope of the invention. Preferably, Form B of 4-[4-(4-fluorophenoxy)benzenesulfonylamino]-tetrahydropyran-4-carboxylic acid hydroxyamide is an anhydrous crystalline form. Form B of 4-[4-(4-fluorophenoxy)benzenesulfonylamino]-tetrahydropyran-4-carboxylic acid hydroxyamide generally has a thermal event associated with melting and decomposition at approximately 190° C. and the following characteristic powder X-ray diffraction pattern with high intensity diffraction peaks at diffraction angles ±0.2 degrees 2θ as follows:

| Angle (2θ) | I*(rel. %) |
|---|---|
| 8.2 | 61 |
| 13 | 91.3 |
| 14.2 | 79.7 |
| 14.7 | 50.2 |
| 15.9 | 46.8 |
| 16.2 | 46.6 |
| 17 | 76.1 |
| 20.6 | 90.8 |
| 24.6 | 100 |
| 25.9 | 74.1 |

The term “treating”, “treat” or “treatment” as used herein includes preventative (e.g., prophylactic) and palliative treatment.

By “pharmaceutically acceptable” it is meant the carrier, diluent, excipients, and/or salt must be compatible with the other ingredients of the formulation, and not deleterious to the recipient thereof.

One of ordinary skill in the art will appreciate that Form B and/or Form A of 4-[4-(4-fluorophenoxy)benzenesulfonylamino]-tetrahydropyran-4-carboxylic acid hydroxyamide are useful in treating a diverse array of diseases. One of ordinary skill in the art will also appreciate that when using Form B and/or Form A in the treatment of a specific disease, they may be combined with various existing therapeutic agents used for that disease. Thus, Form B and/or Form A may be used in combination therapy with standard non-steroidal anti-inflammatory drugs (NSAID'S) and analgesics, and in combination with cytotoxic drugs such as adriamycin, daunomycin, cis-platinum, etoposide, taxol, taxotere and other alkaloids, such as vincristine, in the treatment of cancer.

For the treatment of rheumatoid arthritis, Form B and/or Form A may be combined with agents such as TNF-alpha inhibitors such as anti-TNF monoclonal antibodies and TNF receptor immunoglobulin molecules (such as Enbrel™), low dose methotrexate, lefunimide, hydroxychloroquine, d-penicilamine, auranofin or parenteral or oral gold.

As mentioned above, Form B and/or Form A can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib and rofecoxib, analgesics and intraarticular therapies such as corticosteroids and hyaluronic acids such as hyalgan and synvisc.

In addition, Form B and/or Form A of 4-[4-(4-fluorophenoxy)benzenesulfonylamino]-tetrahydropyran-4-carboxylic acid hydroxyamide may also be used in combination with anticancer agents such as endostatin and angiostatin or cytotoxic drugs such as adriamycin, daunomycin, cis-platinum, etoposide, taxol, taxotere and alkaloids, such as vincristine, and antimetabolites such as methotrexate.

Crystal Form B and/or Form A may also be used in combination with cardiovascular agents such as calcium channel blockers, lipid lowering agents such as statins, fibrates, beta-blockers, Ace inhibitors, Angiotensin-2 receptor antagonists and platelet aggregation inhibitors.

Further, Form B and/or Form A may also be used in combination with CNS agents such as antidepressants (such as sertraline), anti-Parkinsonian drugs (such as deprenyl, L-dopa, requip, miratex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti-Alzheimer's drugs such as Aricept, tacrine, COX-2 inhibitors, propentofylline or metryfonate.

Crystal Form B and/or Form A may also be used in combination with osteoporosis agents such as droloxifene or fosomax and immunosuppressant agents such as FK-506 and rapamycin.

For administration to mammals, including humans, for the inhibition of matrix metalloproteinases or the production of tumor necrosis factor (TNF), a variety of conventional routes may be used including orally, parenterally and topically. In general, Form B and/or Form A of 4-[4-(4-fluorophenoxy)benzenesulfonylamino]-tetrahydropyran-4-carboxylic acid hydroxyamide will be administered orally or parenterally at dosages between about 0.1 and 25 mg/kg body weight of the subject to be treated per day, preferably from about 0.3 to 5 mg/kg. However, some variation in dosage will necessarily occur depending on the condition of the subject being treated. The person responsible for administration will, in any event, determine the appropriate dose for the individual subject.

Crystal Form B and/or Form A can be administered in a wide variety of different dosage forms, in general, the therapeutically effective compounds of this invention are present in such dosage forms at concentration levels ranging from about 5.0% to about 70% by weight.

As noted above, the compositions of the present invention may be prepared in tablet, powder (e.g., capsule) suspension or solution form. If the composition is to be delivered in a tablet form, the tablet may be prepared by wet or dry granulation, or by direct compression. Direct compression techniques are generally known in the art of pharmaceutical science. For example, Form B and/or Form A of 4-[4-(4-fluorophenoxy)benzenesulfonylamino]-tetrahydropyran-4-carboxylic acid hydroxyamide maybe admixed with dry excipients and compressed into tablets.

Dry granulation techniques are generally also known in the art of pharmaceutical science. For example, Form B and/or Form A of 4-[4-(4-fluorophenoxy)benzenesulfonylamino]-tetrahydropyran-4-carboxylic acid hydroxyamide maybe admixed with dry excipients and compressed into large slugs or roller compacted into ribbon-like strands. The compacted material is then suitably milled to produce a free flowing powder which is then compressed into tablets. Additional excipients may then be added and mixed with the free flowing powder before being compressed into tablets. The mixture may be suitable compressed using a single punch or rotary tablet machine. Examples of excipients include calcium phosphate, microcrystalline cellulose, sodium starch glycollate and magnesium stearate which may be admixed in appropriate ratios. Alternatively, a granulation containing Form B and/or Form A of 4-[4-(4-fluorophenoxy)benzenesulfonylamino]-tetrahydropyran-4-carboxylic acid hydroxyamide may be prepared by the procedure described in Ghebre-Sellassie, et al., U.S. Pat. No. 6,499,984, the disclosure of which is incorporated herein by reference in its entirety.

### DESCRIPTION OF THE DRAWINGS

FIG. 1 is a powder X-ray diffraction pattern for Form A of 4-[4-(4-fluorophenoxy)benzenesulfonylamino]-tetrahydropyran-4-carboxylic acid hydroxyamide.

FIG. 2 is a powder X-ray diffraction pattern for Form B of 4-[4-(4-fluorophenoxy)benzenesulfonylamino]-tetrahydropyran-4-carboxylic acid hydroxyamide.

FIG. 3 is a calculated X-ray diffraction pattern for Form B of 4-[4-(4-fluorophenoxy)benzenesulfonylamino]-tetrahydropyran-4-carboxylic acid hydroxyamide.

FIG. 4 is a diagram of ¹³C solid state NMR spectra for polymorph Form A of 4-[4-(4-fluorophenoxy)benzenesulfonylamino]-tetrahydropyran-4-carboxylic acid hydroxyamide.

FIG. 5 is a diagram of ¹³C solid state NMR spectra for polymorph Form B of 4-[4-(4-fluorophenoxy)benzenesulfonylamino]-tetrahydropyran-4-carboxylic acid hydroxyamide.

FIG. 6 is a DSC thermal profile for polymorph Form A of 4-[4-(4-fluorophenoxy)benzenesulfonylamino]-tetrahydropyran-4-carboxylic acid hydroxyamide.

FIG. 7 is a DSC thermal profile for polymorph Form B of 4-[4-(4-fluorophenoxy)benzenesulfonylamino]-tetrahydropyran-4-carboxylic acid hydroxyamide.

The present invention is illustrated by the following Examples and analyses, but it is not limited to the details thereof.

### EXAMPLES

### Preparation of Form A of 4-[4-(4-Fluorophenoxy)Benzenesulfonylamino]-Tetrahydro Pyran-4-Carboxylic Acid Hydroxyamide

The compound 4-[4-(4-fluorophenoxy)benzenesulfonylamino]-tetrahydropyran-4-carboxylic acid hydroxyamide can generally be prepared by the methods disclosed in Reiter, U.S. Pat. No. 6,087,392. Form A may be prepared by Methods A and B below.

Method A

A solution of 4-[4-(4-fluorophenoxy)benzenesulfonylamino]tetrahydropyran-4-carboxylic acid N-benzyloxyamide (11.28 grams, 0.0225 mole) in ethyl acetate (600 ml) was treated with 5% palladium on barium sulfate (5.0 grams) and hydrogenated in a Parr™ shaker at 3 atmospheres pressure for 18 hours. After filtration through nylon (pore size 0.45 mm) to remove the catalyst, the filter pad was rinsed with methanol. Combined filtrate and rinse were evaporated and the residue taken up in hot methanol. Cooling afforded crude 4-[4-(4-fluorophenoxy)benzenesulfonylamino]-tetrahydropyran-4-carboxylic acid hydroxyamide (5.941 grams, 64%, mp 176-177° C.) as white crystalline solids. The mother liquor was evaporated and the residue crystallized from 50% methano/dichloromethane to give additional 4-[4-(4-fluorophenoxy)benzenesulfonylamino]-tetrahydropyran-4-carboxylic acid hydroxyamide (0.660 grams, mp 184-185° C.) as white needles. The mother liquor was again evaporated and the residue crystallized from methanol/dichloromethane to give additional product (1.861 grams, mp 176-177° C.). Recrystallization of the first lot from methanol/dichloromethane provided analytically pure 4-[4-(4-fluorophenoxy)benzenesulfonylamino]tetrahydropyran-4-carboxylic acid hydroxyamide (3.091 grams, mp 184-185° C.).

Method B

Oxalyl chloride (11.83 grams, 0.0932 mole, 1.1 eq.) and DMF (0.13 mL) were added to a stirred suspension of the carboxylic acid (33.25 grams, 0.0841 mole) in dry methylene chloride (300 mL) at room temperature. Some bubbling was observed. The suspension, which slowly became a yellowish solution was stirred overnight at room temperature. Meanwhile, a solution of hydroxylamine hydrochloride (7.65 grams, 0.110 mole, 1.3 eq.) in dry pyridine (51.4 mL, 0.635 mole, 7.5 eq.) at 0° C. was treated with chlorotrimethylsilane causing a white precipitate to form. This suspension was stirred at room temperature overnight. Both flasks were then cooled to 0° C. and the solution of acid chloride was added to the suspension of silylated hydroxylamine. The resulting mixture was stirred at 0° C. for 1 hour and room temperature for 2 hours. Added 1000 mL aqueous 2N HCI and stirred at room temperature for 1 hour. The layers were separated, the aqueous layer was extracted three times with ethylacetate (500 ml). Combined organic layers were washed with water and brine and dried over magnesium sulfate, filtered and the volume of the filtrate reduced to 300 mL at which point a large amount of white crystalline solid had precipitated. This was cooled overnight in a refrigerator. The solid was collected by vacuum filtration, rinsed with cold 1:1 ethylacetate/hexane and dried under high vacuum to give 30.311 grams of the desired hydroxamic acid (87.8%) as a white crystalline solid (mp 189-190° C.). The following information was obtained from analysis of Form A.

HNMR (d₆ DMSO) & 10.35 (br s, 1H), 8.68 (br s, 1H), 7.78 (br s, 1H), 7.74 (d, 2H), 7.26 (t, 2H), 7.16 (m, 2H), 7.04 (d, 2H), 3.40 (m, 2H), 3.31 (m, 2H), 1.78 (m, 4H). ¹³CNMR (DMSO) & 169.65, 160.66, 137.50, 129.39, 122.34, 122.25, 117.75, 117.44, 117.24, 62.94, 58.45, 33.34. MS Atmospheric Pressure Chemical Ionization Mass Spectra: 409 (M+−1) (ion).

Bulk Preparation of Form A and Form B

Crystal Form B of 4-[4-(4-fluorophenoxy)benzenesulfonylamino]tetrahydropyran-4-carboxylic acid hydroxyamide can generally be prepared directly from the free base solution or by conversion from Form A. For example, Form B may be prepared by seeding the free base solution with Form B seed crystals, or by recrystallization/reslurry of Form A in various solvents. Suitable recrystallization solvents include polar and organic solvents such as methanol, acetone, THF, isopropanol, or ethanol or combinations of such solvents.

In preparing Form A, approximately 385 gallons (1,457 liters) of a compound of formula (I)/ethyl acetate solution (80 volumes of ethyl acetate, for every 1 gram of 4-[4-(4-fluorophenoxy)benzenesulfonylamino]tetrahydropyran-4-carboxylic acid hydroxyamide there was 80 mls of ethyl acetate) was vacuum concentrated with a reaction temperature of 15° C. to 30° C. to a volume of 12 gallons (45.4 liters) to 16 gallons (61.6 liters). Reaction mixture heterogeneous off-white colored slurry with substantial solids precipitated out of solution. At a reaction concentration of approximately three volumes and a reaction temperature of −3° C. to 2° C. 4-[4-(4-fluorophenoxy)benzenesulfonylamino]tetrahydropyran-4-carboxylic acid hydroxyamide polymorph Form A was isolated from ethyl acetate. The polymorph Form A of 4-[4-(4-fluorophenoxy)benzenesulfonylamino]tetrahydropyran-4-carboxylic acid hydroxyamide solids were then washed with 3.8 volumes (18.4 gallons total) of cold, −3° C. to 2° C., ethyl acetate: hexanes (1:1). 4-[4-(4-fluorophenoxy)benzenesulfonylamino]tetrahydropyran-4-carboxylic acid hydroxyamide polymorph Form A was then vacuum dried with nitrogen bleed at 38° C. to 42° C. This resulted in 13.9 kilograms of 4-[4-(4-fluorophenoxy)benzenesulfonylamino]tetrahydropyran-4-carboxylic acid hydroxyamide polymorph Form A material.

Form A of 4-[4-(4-fluorophenoxy)benzenesulfonylamino]tetrahydropyran-4-carboxylic acid hydroxyamide, a total of 28.54 kilograms, were dissolved in 45 volumes methanol. The methanol was then displaced with 2B ethanol and concentrated under vacuum, filtered, dried and milled. The product is Form B.

The bulk polymorph conversion of 4-[4-(4-fluorophenoxy)benzenesulfonylamino]tetrahydropyran-4-carboxylic acid hydroxyamide from Form A to Form B was carried out as follows. A total of 26.4 kilograms of 4-[4-(4-fluorophenoxy)benzenesulfonylamino]tetrahydropyran-4-carboxylic acid hydroxyamide Form A was reslurried in eight volumes of 2B ethanol seeding and stirring for 24 to 30 hours at 20° C.-25° C. The material was then filtered, washed, and dried. The procedure resulted 25.9 kilograms of 4-[4-(4-fluorophenoxy)benzenesulfonylamino]tetrahydropyran-4-carboxylic acid hydroxyamide polymorph Form B with a yield of 98%.

The following analyses were conducted and data were determined for Form A and Form B.

Powder X-ray Diffraction analyses of Form A and Form B

Powder X-ray diffraction analyses were conducted according to methods known in the art and under the following conditions: a Cu anode was used; wavelength 1: 1.54056; wavelength 2: 1.54439 (Rel Intensity: 0.500); range #1-coupled: 3.000 to 40.000; step size: 0.040; step time: 1.00; smoothing width: 0.300; and threshold: 1.0.

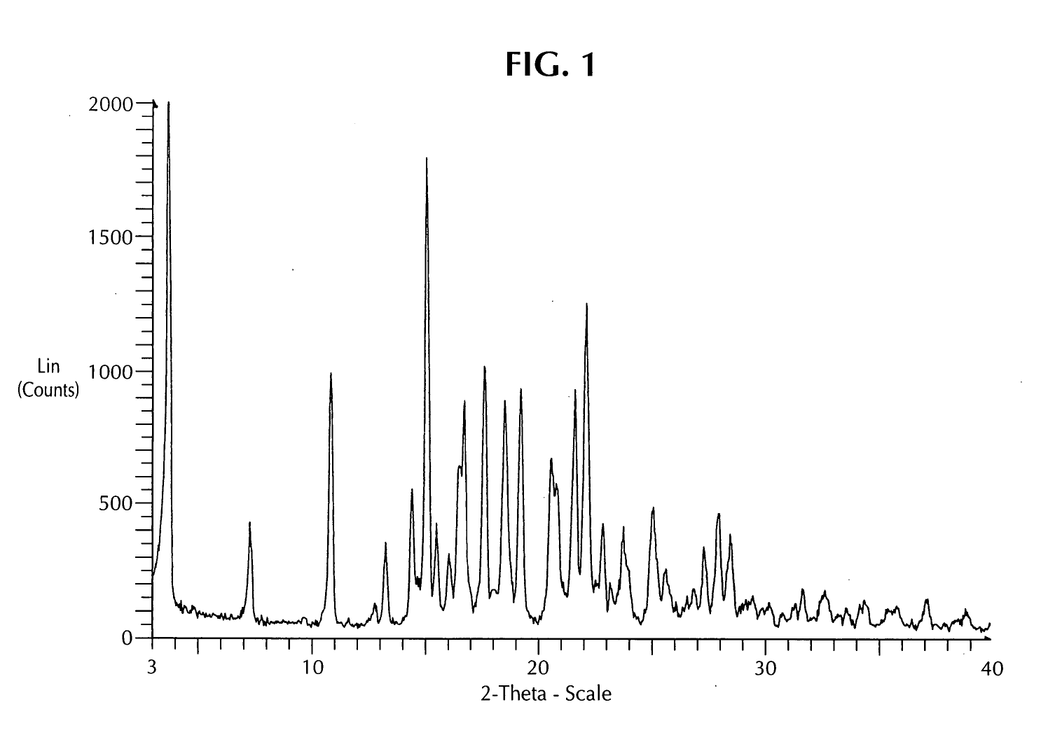

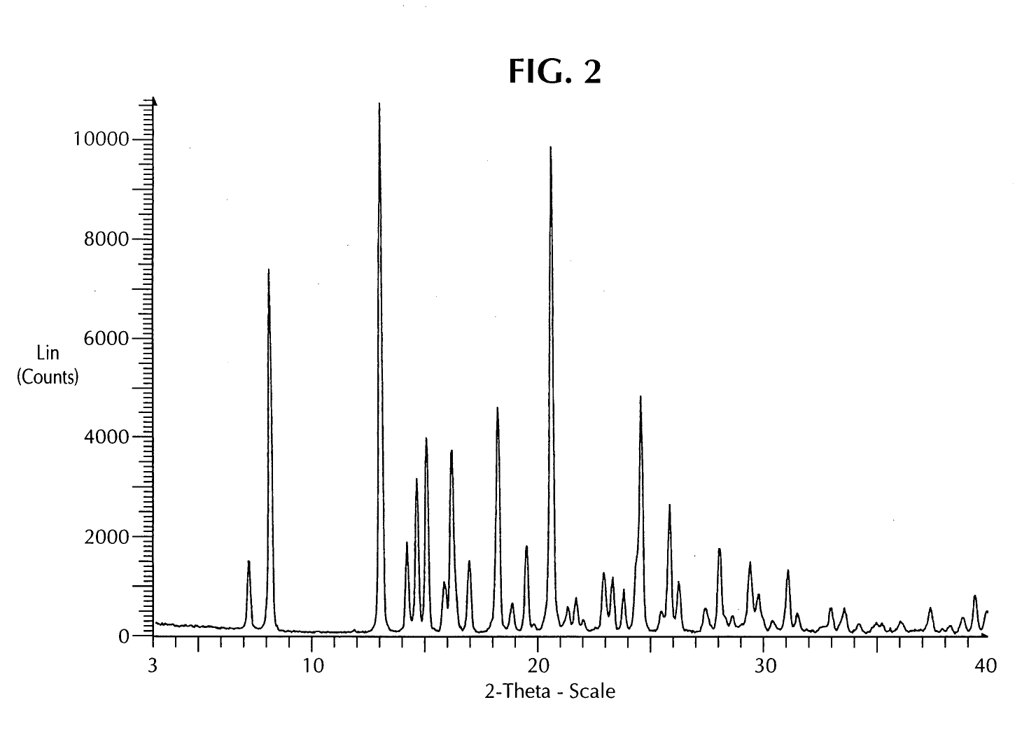

Powder X-ray Diffraction Patterns were produced for Form A (see FIG. 1) and Form B (see FIG. 2). The peak lists corresponding to each of the patterns are given in Table 1 and 2 below.

TABLE 1
Form A - PXRD pattern peak list (2-theta ± 0.2 degrees)

| Angle | I* |
|---|---|
| 2 theta | (rel. %) |
| 3.6 | 100.0 |
| 7.2 | 21.2 |
| 10.8 | 49.8 |
| 12.7 | 6.4 |
| 13.2 | 17.7 |
| 14.4 | 27.8 |
| 15.0 | 90.1 |
| 15.5 | 21.4 |
| 16.0 | 15.7 |
| 16.5 | 32.2 |
| 16.6 | 44.8 |
| 17.6 | 51.5 |
| 18.5 | 44.9 |
| 19.2 | 47.2 |
| 20.6 | 33.9 |
| 20.8 | 29.0 |
| 21.6 | 47.2 |
| 22.1 | 63.5 |
| 22.8 | 21.7 |
| 23.2 | 10.7 |
| 23.7 | 21.2 |
| 25.1 | 24.8 |
| 25.6 | 13.3 |
| 26.6 | 8.2 |
| 26.9 | 9.6 |
| 27.3 | 17.4 |
| 28.0 | 23.8 |
| 28.5 | 19.9 |
| 29.2 | 7.7 |
| 29.5 | 8.5 |
| 30.3 | 7.1 |
| 31.4 | 7.1 |
| 31.8 | 9.7 |
| 32.7 | 9.5 |
| 33.7 | 6.4 |
| 34.3 | 7.1 |
| 34.5 | 7.8 |
| 35.4 | 6.4 |
| 35.9 | 6.9 |
| 37.2 | 8.1 |
| 38.9 | 6.5 |

*The relative intensities may change depending on the crystal size and morphology.

TABLE 2
Form B - PXRD pattern peak list (2-theta ± 0.2 degrees)

| Angle | I* |
|---|---|
| 2-theta | (rel. %) |
| 7.2 | 13.9 |
| 8.1 | 68.7 |
| 13.1 | 100.0 |
| 14.2 | 17.8 |
| 14.7 | 29.6 |
| 15.1 | 37.2 |
| 15.9 | 10.4 |
| 16.2 | 34.9 |
| 16.9 | 14.4 |
| 18.2 | 43.1 |
| 18.8 | 6.5 |
| 19.5 | 17.1 |
| 20.6 | 92.0 |
| 21.4 | 5.9 |
| 21.7 | 7.6 |
| 22.0 | 3.4 |
| 22.9 | 12.3 |
| 23.3 | 11.5 |
| 23.8 | 9.2 |
| 24.6 | 45.7 |
| 25.5 | 5.1 |
| 25.9 | 25.2 |
| 26.3 | 10.7 |
| 27.5 | 5.7 |
| 28.1 | 16.9 |
| 28.7 | 4.4 |
| 29.5 | 14.5 |
| 29.8 | 8.5 |
| 30.5 | 3.4 |
| 31.2 | 13.1 |
| 31.6 | 5.0 |
| 32.7 | 2.5 |
| 33.0 | 6.0 |
| 33.7 | 6.1 |
| 34.3 | 3.1 |
| 35.0 | 3.4 |
| 35.3 | 3.3 |
| 35.6 | 2.4 |
| 36.1 | 3.7 |
| 37.4 | 6.2 |
| 38.3 | 2.8 |
| 38.8 | 4.3 |
| 39.4 | 8.6 |

*The relative intensities may change depending on the crystal size and morphology.

Single Crystal X-Ray Analysis

The crystal structure for Form B was determined by X-ray analysis of a single crystal of Form B produced by the method described herein. Data was collected at room temperature using Bruker X-ray diffractometers equipped with copper radiation and graphite monochromators. Structures were solved using direct methods. The SHELXTL computer library provided by Bruker AXS, Inc facilitated all necessary crystallographic computations and molecular displays. The results are displayed in Table 3 below.

TABLE 3
Single Crystal Data For Form B

| | Form B |
|---|---|
| Empirical formula | C₁₈H₁₉N₂O₆SF |
| Formula weight | 410.4 |
| Crystal size (mm) | 0.04 × 0.20 × 0.24 |
| Space group | P2₁/c monoclinic |
| Unit cell dimensions | a = 6.250 (2) Å |
| | b = 24.548 (5) Å |
| | c = 12.132 (2) Å |
| | α = 90° |
| | β = 95.01(2)° |
| | γ = 90° |
| Z (per formular) | 4 |
| Density (g/cm³) | 1.470 |
| R | 0.0469 |

Calculation of PXRD Pattern from Single Crystal Data

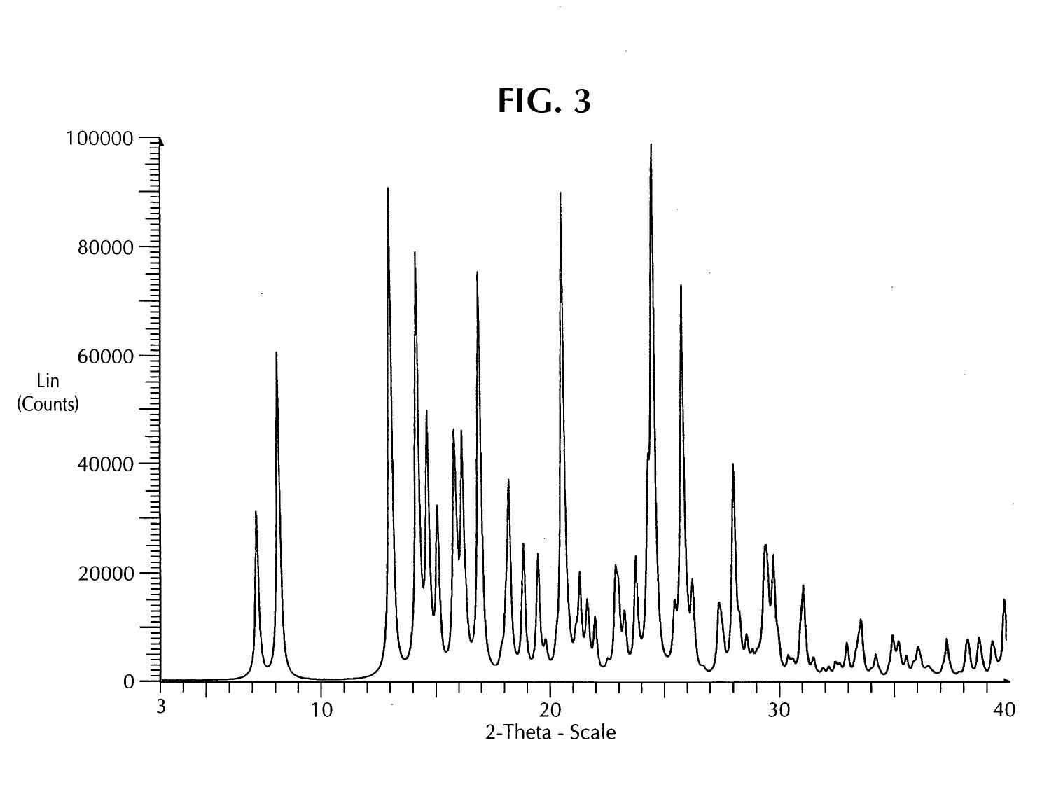

The single crystal structural data provide the cell dimensions, space group and atomic positions of a crystal form. According to methods known in the arts, these parameters were used as the basis to calculate a “perfect” powder pattern of that crystal form. The calculation can be done using SHELXTL Plus computer program, Reference Manual by Siemens Analytical X-ray Instrument, Chapter 10, p. 179-181, 1990. Comparing the calculated PXRD pattern (see FIG. 3) and the experimental powder pattern (see FIG. 2) will confirm whether a powder sample corresponds to an assigned single crystal structure. This procedure has been performed on the crystal Form B and a match between the two patterns indicates the agreement between powder sample and the corresponding single crystal structure. A peak list corresponding to the calculated PXRD pattern is provided in Table 4 below.

TABLE 4
Form B PXRD pattern peak list

| Angle | I* |
|---|---|
| 2-theta | (rel. %) |
| 7.2 | 31.3 |
| 8.2 | 61.0 |
| 13.0 | 91.3 |
| 14.2 | 79.7 |
| 14.7 | 50.2 |
| 15.1 | 32.7 |
| 15.9 | 46.8 |
| 16.2 | 46.6 |
| 17.0 | 76.1 |
| 18.2 | 37.6 |
| 18.9 | 25.7 |
| 19.5 | 23.9 |
| 19.8 | 8.2 |
| 20.6 | 90.8 |
| 21.3 | 20.6 |
| 21.7 | 15.5 |
| 22.0 | 12.3 |
| 22.6 | 4.6 |
| 23.0 | 21.9 |
| 23.3 | 13.5 |
| 23.8 | 23.6 |
| 24.3 | 42.6 |
| 24.6 | 100.0 |
| 25.5 | 15.5 |
| 25.9 | 74.1 |
| 26.3 | 19.3 |
| 27.5 | 15.0 |
| 28.1 | 40.7 |
| 28.6 | 9.1 |
| 29.5 | 25.6 |
| 29.8 | 23.7 |
| 30.5 | 5.2 |
| 31.1 | 18.1 |
| 31.5 | 4.8 |
| 32.5 | 4.0 |
| 33.0 | 7.6 |
| 33.6 | 11.9 |
| 34.3 | 5.3 |
| 35.0 | 9.0 |
| 35.3 | 7.9 |
| 35.6 | 5.1 |
| 36.1 | 6.9 |
| 37.4 | 8.4 |
| 38.2 | 8.3 |
| 38.8 | 8.6 |
| 39.3 | 8.0 |

*The calculated powder X-ray diffraction pattern represents all peaks with intensity % > 4%. Peaks in italic were absent in the experimental PXRD pattern due to low intensity or within experimental error of ±0.2 degree 2-theta.

Solid State NMR Analysis:

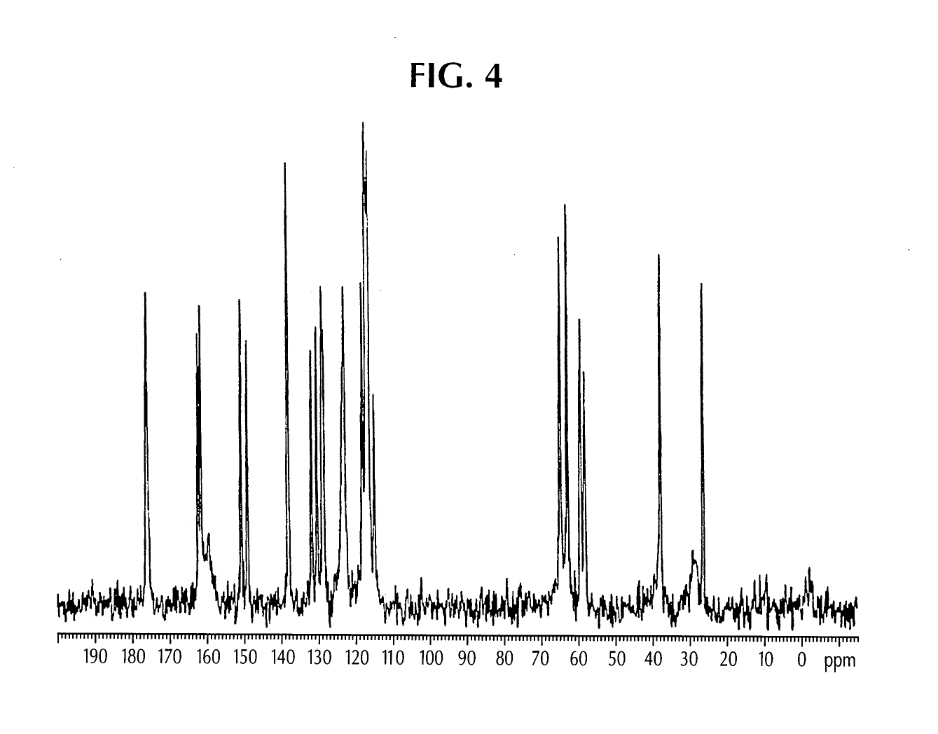

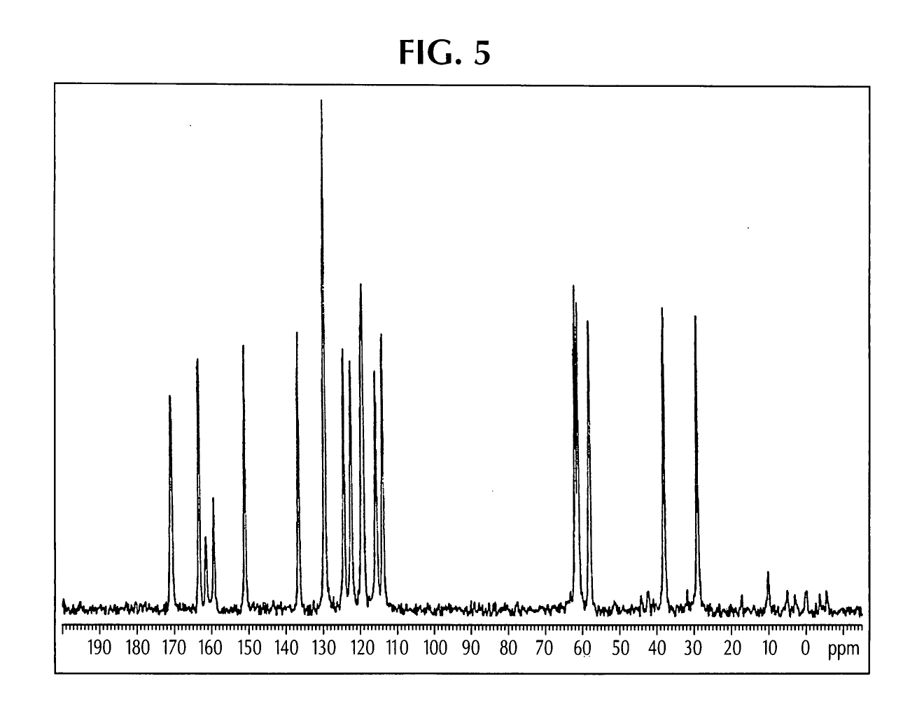

¹³C solid state NMR spectra were collected for Form A (see FIG. 4) and Form B (see FIG. 5) of 4-[4-(4-fluorophenoxy)benzenesulfonylamino]tetrahydropyran-4-carboxylic acid hydroxyamide using a 11.75 T spectrometer (Bruker Biospin, Inc., Billerica, Mass.), corresponding to 125 MHz ¹³C frequency. The spectra were acquired using a cross-polarization magic angle spinning (CPMAS) probe operating at ambient temperature and pressure. 4 mm BL Bruker probes were employed, accommodating 75 mg of sample with maximum speed of 15 kHz. Data was processed with an exponential line broadening function of 5.0 Hz. Proton decoupling of 100 kHz was used. A sufficient number of acquisitions were averaged out to obtain adequate signal-to-noise ratios for all peaks. ¹³C spectrum of form A was acquired using 288 scans with recycle delay of 150 s, corresponding to approximately 12 hour total acquisition time. ¹³C spectrum of form B was acquired using 416 scans with recycle delay of 40 s, corresponding to approximately 4.5 hour total acquisition time. The peaks not included in the peak list are spinning sidebands. Magic angle was adjusted using KBr powder according to standard NMR vendor practices. The spectra were referenced relative to the upfield resonance of adamantane (ADMNT) at 29.5 ppm. The spectral window minimally included the spectra region from 220 to −10 ppm. Table 5 below provides chemical shift data for Form A and Form B that corresponds to FIG. 4 and FIG. 5, respectively.

TABLE 5
¹³C SS-NMR Chemical Shifts in ppm (±0.2 ppm)

| Form A | Form B |
|---|---|
| 175.9 | 171.0 |
| 162.1 | 163.6 |
| 161.5 | 161.8 |
| 159.4 | 159.6 |
| 150.8 | 151.2 |
| 149.1 | 136.8 |
| 138.2 | 129.8 |
| 131.9 | 124.6 |
| 130.5 | 122.6 |
| 129.0 | 119.5 |
| 128.7 | 116.0 |
| 123.0 | 114.2 |
| 118.1 | 62.0 |
| 117.1 | 61.2 |
| 116.4 | 58.1 |
| 114.9 | 37.8 |
| 64.7 | 28.9 |
| 62.8 | |
| 59.2 | |
| 58.1 | |
| 37.7 | |
| 28.2 | |
| 26.3 | |

Thermal analysis of Form A and Form B

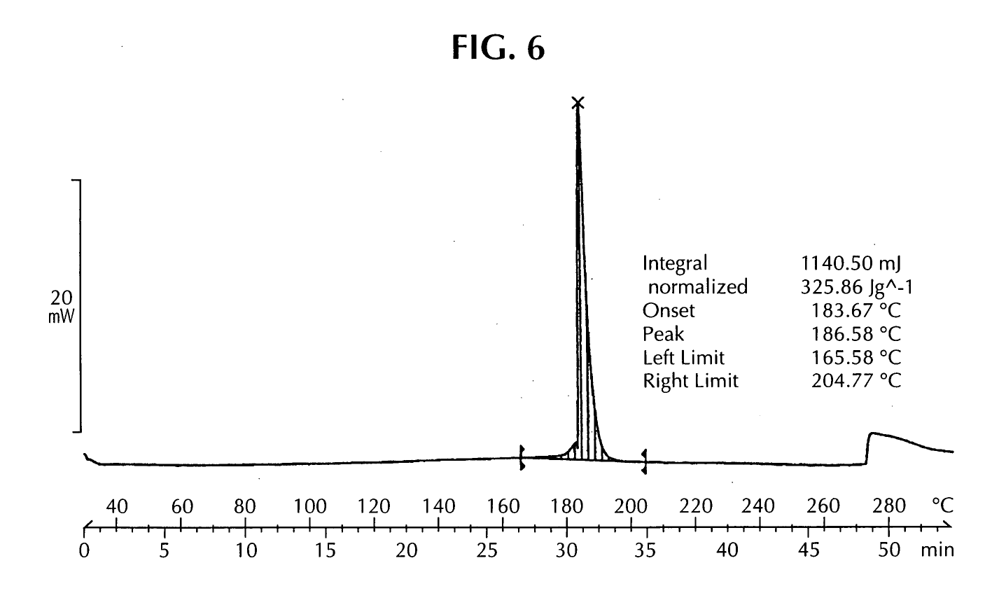

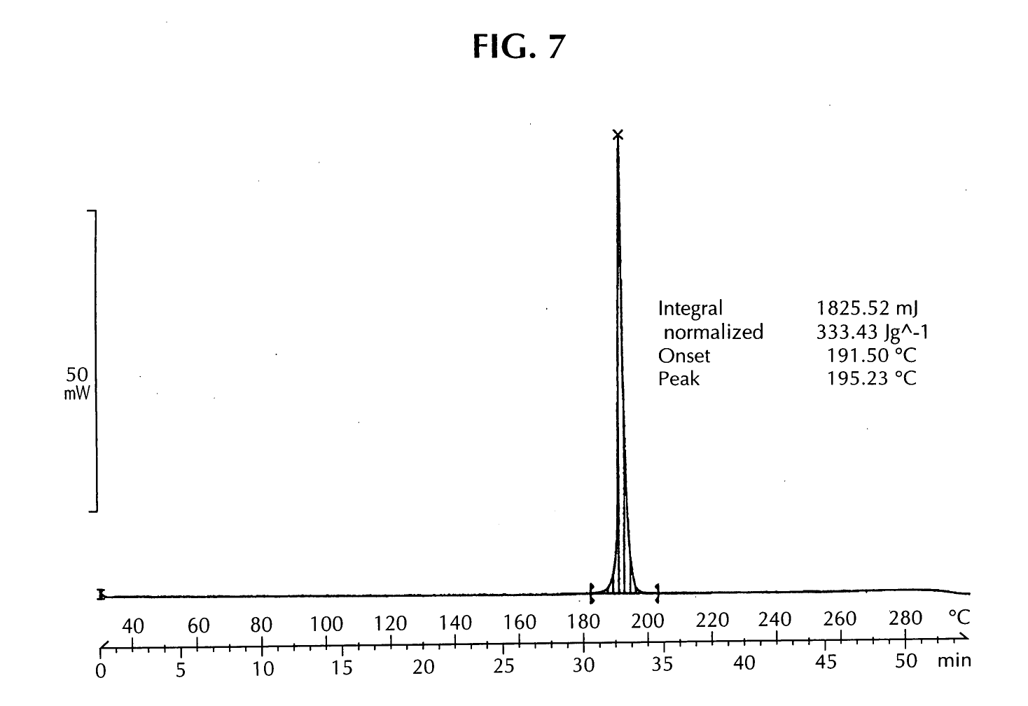

Differential Scanning Calorimetry (DSC) analysis was carried out samples of Form A and Form B using a Mettler DSC 821, calibrated with indium. Each DSC sample was prepared by weighing 2-4 mg of material in an aluminum pan with a pinhole. The sample was heated under nitrogen, at a rate of 5° C. per minute from 30 to 300° C. The onset temperature of the melting endotherm was reported as the melting temperature. The thermal event for Form A was determined to be approximately 184 ° C., while the thermal event for Form B was about 190 ° C. DSC diagrams are provided for Form A (see FIG. 6) and Form B (see FIG. 7).

Although the foregoing invention has been described in some detail by way of illustration and example for purposes of clarity of understanding, it will be obvious that certain changes and modifications may be practiced within the scope of the appended claims.

1. Crystalline Form B of 4-[4-(4-fluorophenoxy)benzenesulfonylamino]-tetrahydropyran-4-carboxylic acid hydroxyamide.

2. Anhydrous crystalline Form B of 4-[4-(4-fluorophenoxy)benzenesulfonylamino]-tetrahydropyran-4-carboxylic acid hydroxyamide.

3. The crystalline form according to claim 1 or 2 having a single crystal X-ray crystallographic analysis with crystal parameters shown as the following:

| | |
|---|---|
| Empirical formula | C₁₈H₁₉N₂O₆SF |
| Space group | P2₁/c monoclinic |
| Unit cell dimensions | a = 6.250 (2) Å |
| | b = 24.548 (5) Å |
| | c = 12.132 (2) Å |
| | α = 90° |
| | β = 95.01(2)° |
| | γ = 90° |

4. The crystalline form according to claim 1 or 2 and having an experimental powder X-ray diffraction pattern having high intensity peaks at 8.2, 13, 14.2, 14.7, 15.9, 16.2, 17, 20.6, 24.6, and 25.9, ±0.2 degrees 2θ.

5. The crystalline form according to claim 1 or 2, wherein said crystal form has a thermal event of approximately 190° C.

6. The crystalline form according to claim 1 or 2, wherein said crystal form has a ¹³CNMR spectra of: 171.0, 163.6, 161.8, 159.6, 151.2, 136.8, 129.8, 124.6, 122.6, 119.5, 116.0, 114.2, 62.0, 61.2, 58.1, 37.8, and 28.9±0.2 ppm.

7. A mixture of crystal form B and crystal form A of 4-[4-(4-fluorophenoxy)benzenesulfonylamino]-tetrahydropyran-4-carboxylic acid hydroxyamide.

8. A mixture according to claim 7 wherein wherein Form B has an experimental powder X-ray diffraction pattern of high intensity peaks at 8.2, 13, 14.2, 14.7, 15.9, 16.2, 17, 20.6, 24.6, and 25.9, ±0.2 degrees 2θ, and Form A has an experimental powder x-ray diffraction pattern of high intensity peaks at 3.6, 10.8, 15.0, 16.6, 17.6, 18.5, 19.2, 21.6 and 22.1±0.2 degrees 2θ.

9. A mixture according to claim 7 wherein Form B has ¹³C ss-NMR chemical shifts at 171.0, 163.6, 161.8, 159.6, 151.2, 62.0, 61.2, 58.1, 37.8 and 28.9, and Form A has ¹³C ss-NMR chemical shifts at 175.9, 162.1, 161.5, 159.4, 150.8, 149.1, 64.7, 62.8, 59.2, 58.1, 37.7, and 26.3±0.2 ppm, and wherein the mixture is in a solid dosage form.

10. A pharmaceutical composition comprising crystalline Form B of 4-[4-(4-fluorophenoxy)benzenesulfonylamino]-tetrahydropyran-4-carboxylic acid hydroxyamide and at least one inert pharmaceutically acceptable carrier or diluent.

11. A pharmaceutical composition comprising anhydrous crystalline Form B of 4-[4-(4-fluorophenoxy)benzenesulfonylamino]-tetrahydropyran-4-carboxylic acid hydroxyamide and at least one inert pharmaceutically acceptable carrier or diluent.

12. A pharmaceutical composition comprising a pharmaceutically effective amount of the crystalline form according to claim 3 together with at least one inert pharmaceutically acceptable carrier or diluent.

13. A pharmaceutical composition comprising a pharmaceutically effective amount of the crystalline form according to claim 4 together with at least one inert pharmaceutically acceptable carrier or diluent.

14. A method of treating an inflammatory disease in a mammal comprising administering to the mammal a therapeutically effective amount of crystalline Form B of 4-[4-(4-fluorophenoxy)benzenesulfonylamino]-tetrahydropyran-4-carboxylic acid hydroxyamide.

15. A method of treating inflammatory disease in a mammal comprising administering to the mammal a therapeutically effective amount of anhydrous crystalline Form B of 4-[4-(4-fluorophenoxy)benzenesulfonylamino]-tetrahydropyran-4-carboxylic acid hydroxyamide.

16. A method of treating inflammatory disease in a mammal comprising administering to the mammal a therapeutically effective amount of the crystalline Form B according to claim 3.

17. A method of treating inflammatory disease in a mammal comprising administering to the mammal a therapeutically effective amount of the crystalline Form B according to claim 4.

18. A process for preparing crystalline Form B of 4-[4-(4-fluorophenoxy)benzenesulfonylamino]-tetrahydropyran-4-carboxylic acid hydroxyamide comprising preparing a solution containing 4-[4-(4-fluorophenoxy)benzenesulfonylamino]-tetrahydropyran-4-carboxylic acid hydroxyamide and seeding the solution with a crystal of Form B of 4-[4-(4-fluorophenoxy)benzenesulfonylamino]-tetrahydropyran-4-carboxylic acid hydroxyamide.

19. A process for preparing crystalline Form B of 4-[4-(4-fluorophenoxy)benzenesulfonylamino]-tetrahydropyran-4-carboxylic acid hydroxyamide comprising preparing Form A of 4-[4-(4-fluorophenoxy)benzenesulfonylamino]-tetrahydropyran-4-carboxylic acid hydroxyamide and recrystallizing the Form A product to obtain Form B of 4-[4-(4-fluorophenoxy)benzenesulfonylamino]-tetrahydropyran-4-carboxylic acid hydroxyamide.
